(19) United States
(12) Patent Application Publication
(10) Pub. No.: US 2003/0225182 A1
(43) Pub. Date: Dec. 4, 2003

(54) Dental composition with improved light stability

(75) Inventors: Uwe Walz, (US); Joachim E. Klee, (US)

Correspondence Address: Douglas J. Hura, DENTSPLY INTERNATIONAL INC., 570 West College Avenue, York, PA, 17405-0872, US

(73) Assignee: DENTSPLY DeTrey GmbH

(21) Appl. No.: 10/452,739

(22) Filed: Jun. 2, 2003

Related U.S. Application Data
(63) Continuation of application No. 09/754,162, filed on Jan. 4, 2001, now abandoned.

(51) Int. Cl. A61K 6/08

(52) U.S. Cl. 523/116

(57) ABSTRACT

Dental composition having an improved light and thermal stability, including a mixture of at least a polymerizable resin, at least a polymerizable monomer, at least a polymerization initiator and/or a sensitizer and stabilizer, and at least an organic and/or inorganic filler and pigments in a content of 0 to 90 percent and at least one of the stable radicals.

Claimed is a light curing dental composition with improved light sensitivity comprising prepolymers, macromonomers or polymers having at least one N-1-oxyl moiety, preferably a 4-Amino-2,2,6,6-tetramethylpiperidin-1oxyl moiety.

### TECHNICAL BACKGROUND

Dental compositions comprise polymerizable acrylates and/or methacrylates that are stabilized against spontaneous polymerization by using of free-radical scavenger such as the well-known phenols 2,6-di-tert.-butyl-4-cresol (BHT), hydroquinone or hydroquinone monomethylether (HQME). On the other side they contains a photoinitiator that must be react sensible to visible or UV-light to photoinitiate the free-radical polymerization.

Light curing dental materials mostly are applied under the conditions of relatively strong operating lamps. Consequently, the international standards require that a dental composite remains stable under an illumination of 10,000 lux for 60 seconds (ISO 4049), that a dental pit and fissure sealant and a light activated water based cement remains stable under an illumination of 8,000 lux for 25 seconds (ISO 6874) and for 30 s (ISO 9917-2), respectively.

To improve light stability an optimization of the initiator/inhibitor system leads to lengthening the working times under the conditions of a dental practice. However, this optimization is limited and leads to minor reduction of light sensitivity only.

Recently, it was found, that stable organic radicals reduce the light sensitivity of a dental light-curing composite material (N. Moszner, V. Rheinberger, U.S. Pat. No. 5,847,025) when low molecular stable radicals such as 2,2-Diphenyl-1-picrylhydrazyl radicals, galvinoxyl radicals and/or triphenylmethyl radicals or 2,2,6,6-tetramethylpiperidin-1 oxyl radicals are applied.

In the last decades dental composites becomes popularly as consequence of an improved dental supply. However, the application of this material class is combined with some new risks due to the release of parts of the composite, namely partly non-polymerized monomers (L. Shajii, J. P. Santerre, Biomaterials 20 (1999) 1897, W. R. Hume, T. M. Gerzia, Crit. Rev. Oral. Biol. Med. 7 (1996) 172) as well as portions of the inhibitors and/or initiator system (P. A. Liso et al., Biomaterials 18 (1997) 15). Furthermore, it is well known that free-radicals bearing some health risk (A. T. Diplock et al., Br. J. Nutr. 80 (1998), Suppl 1, 77; L. U. Thompson, Crit. Rev. Food Sci. Nutr. 34 (1994), 473).

Consequently, it seems desirable to use stable free-radicals for improved light sensitivity and to link them into the polymer system in order to avoid penetration and health risks.

The low molecular stable radicals that are suggested in U.S. Pat. No. 5,847,025 bases on piperidinium 1-oxyl radicals bearing phenol or thiophenol groups or derivatives of carboxylic or thiocarboxylic acids.

### DESCRIPTION OF THE INVENTION

Invented was a dental composition having an improved light and thermal stability, comprising a mixture of

(i) at least a polymerizable resin

(ii) at least a polymerizable monomer

(iii) at least a polymerization initiator and/or a sensitizer and stabilizer

(iv) at least an organic and/or inorganic filler and pigments in a content of 0 to 90 percent

(v) and at least one of the stable radicals of formulas 1 to 5

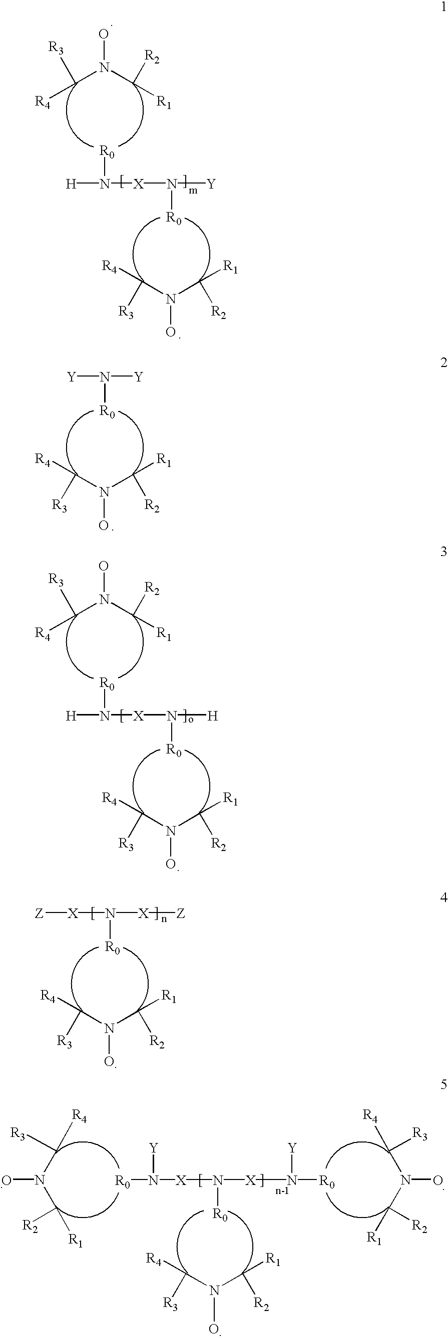

wherein

R₀ denotes a substituted or unsubstituted C₁ to C₁₈ alkylene,

R₁, R₂, R₃ and R₄ denotes a substituted or unsubstituted C₁ to C₁₈ alkylene, preferably a methyl group

X denotes a difunctional substituted or unsubstituted C₂ to C₃₀ alkylene, C₅ to C₃₀ substituted or unsubstituted cycloalkylene, substituted or unsubstituted C₅ to C₃₀ arylene or heteroarylene, preferably the following structures

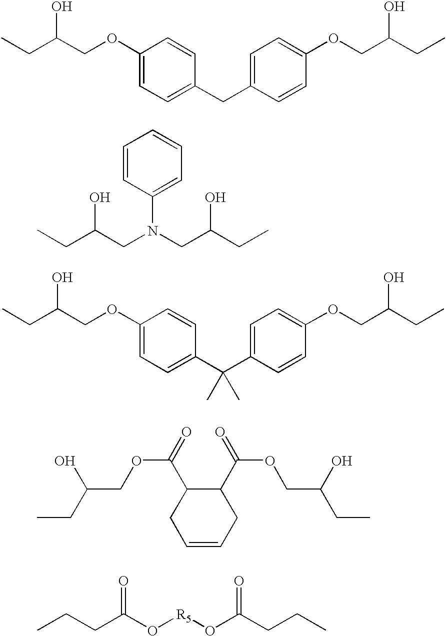

wherein R₅ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,

Y denotes H or a monofunctional substituted or unsubstituted C₁ to C₁₅ alkyl, C₅ to C₁₈ substituted or unsubstituted cycloalkyl, substituted or unsubstituted C₅ to C₁₈ aryl or heteroaryl, preferably selected from the group

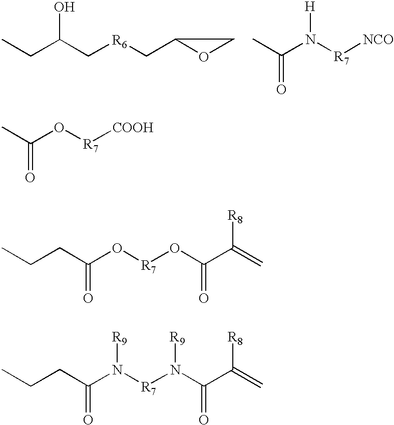

wherein

R₆ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably

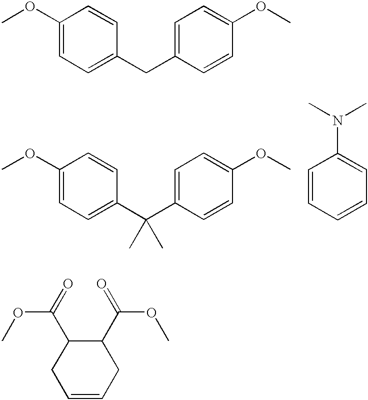

R₇ denotes difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably selected from the group

R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

R₉ denotes a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

Z denotes hydrogen, or a polymerizable moiety, preferably selected from the group of

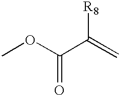

wherein

R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

n, m and o are integers.

Preferably the dental composition comprises at least one of the compounds 6 to 10 which having at least one piperidinium nitroxyl radical moiety

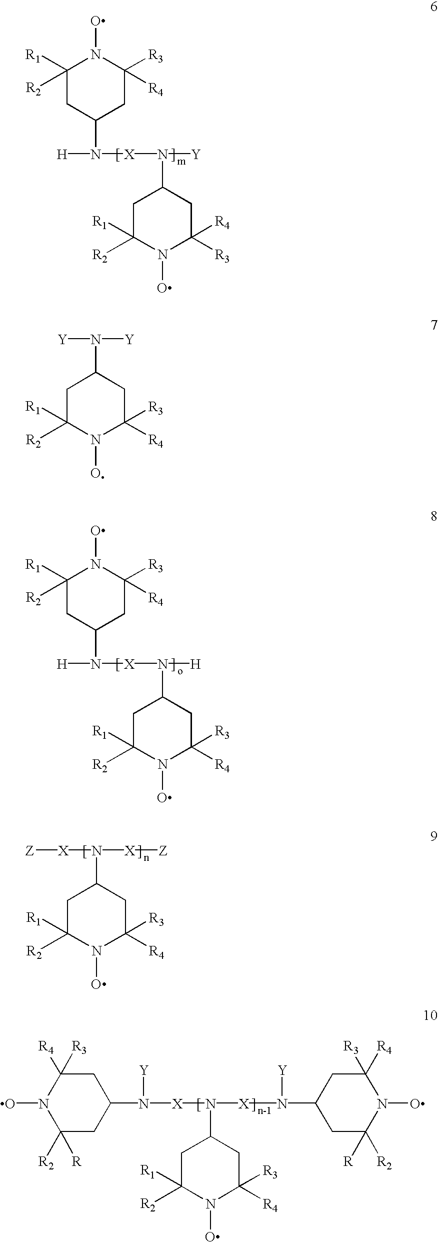

wherein

R₁, R₂, R₃ and R₄ denotes a substituted or unsubstituted C₁ to C₁₈ alkylene, preferably methyl group

X denotes a difunctional substituted or unsubstituted C₂ to C₃₀ alkylene, C₅ to C₃₀ substituted or unsubstituted cycloalkylene, substituted or unsubstituted C₅ to C₃₀ arylene or heteroarylene, preferably the following structures

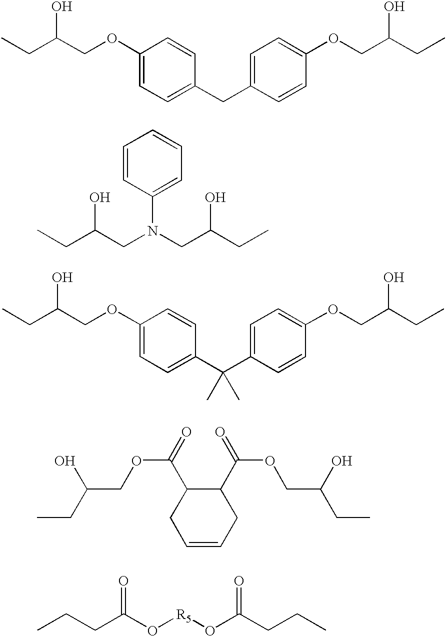

wherein R₅ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,

Y denotes H or a monofunctional substituted or unsubstituted C₁ to C₁₈ alkyl, C₅ to C₁₈ substituted or unsubstituted cycloalkyl, substituted or unsubstituted C₅ to C₁₈ aryl or heteroaryl, preferably selected from the group

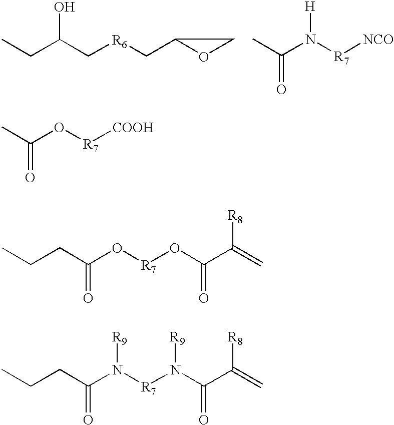

wherein

R₆ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably

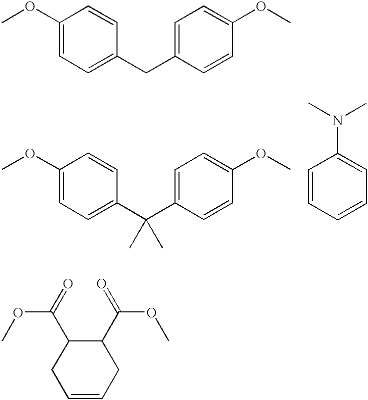

R₇ denotes difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably selected from the group

R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

R₉ denotes a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

Z denotes hydrogen, or a polymerizable moiety, preferably selected from the group of

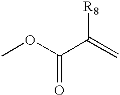

wherein

R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

n, m and o are integers.

The piperidinium nitroxyl radical moieties were obtained by two different pathways, namely by oxidation of the following compounds 11 to 15 or by incorporation of an amine comprising at least a nitroxyl radical moieties.

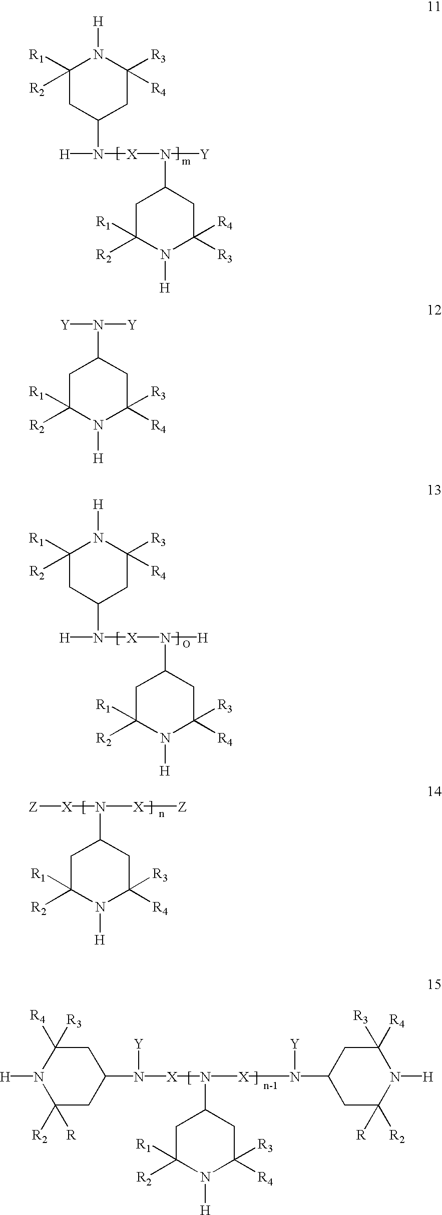

wherein

R₁, R₂, R₃ and R₄ denotes a substituted or unsubstituted C₁ to C₁₈ alkylene, preferably a methyl group

X denotes a difunctional substituted or unsubstituted C₂ to C₃₀ alkylene, C₅ to C₃₀ substituted or unsubstituted cycloalkylene, substituted or unsubstituted C₅ to C₃₀ arylene or heteroarylene, preferably the following structures

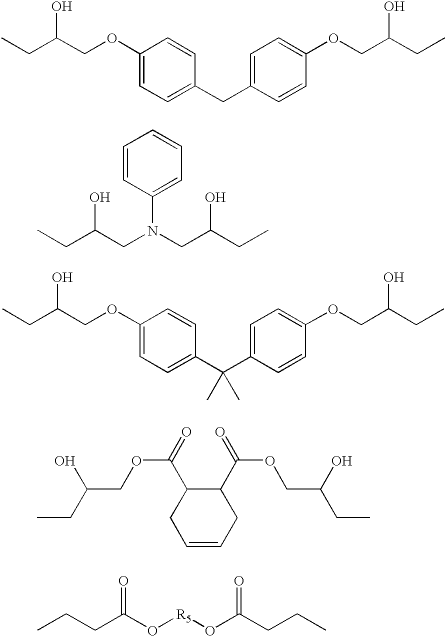

wherein R₅ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,

Y denotes H or a monofunctional substituted or unsubstituted C₁ to C₁₈ alkyl, C₅ to C₁₈ substituted or unsubstituted cycloalkyl, substituted or unsubstituted C₅ to C₁₈ aryl or heteroaryl, preferably selected from the group

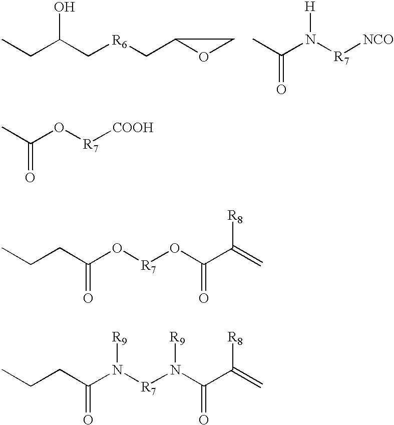

wherein

R₆ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably

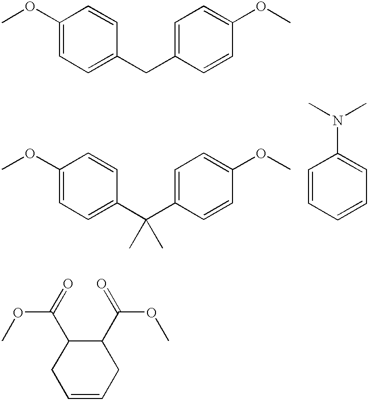

R₇ denotes difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably selected from the group

R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

R₉ denotes a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

Z denotes hydrogen, or a polymerizable moiety, preferably selected from the group of

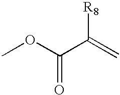

wherein

R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene

n, m and o are integers.

Furthermore, polymers, prepolymers or macromonomers comprising at least a nitroxyl radical moieties were synthesized by direct incorporation of amines 16 comprising at least a nitroxyl radical moieties

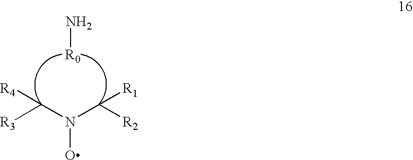

wherein

R₀ denotes a substituted or unsubstituted C₁ to C₁₈ alkylene,

R₁, R₂, R₃ and R₄ denotes a substituted or unsubstituted C₁ to C₁₈ alkylene, preferably methyl group

with a molecule of group A, selected from the group of a diepoxide, a diisocyanate, a dicarboxylic acid or a derivative thereof, a bisacrylamide or a bisacrylate or

with a molecule of group B, selected from the group of molecules that comprise at least an epoxide and a methacrylate group, an epoxide and an isocyanate, a methacrylate and an isocyanate group, an acrylate and a methacrylate group, or with a mixture of molecules A and B.

Amines containing at least a nitroxyl radical moieties are used as comonomers for synthesis of polyamides, polyamidoamines, polyesteramines, polyureas, epoxide-amine addition polymers or prepolymers or macromonomers with the corresponding structural units mentioned above.

Preferably compounds 17 and 18 were use comprising a piperidinium nitroxyl radical moiety.

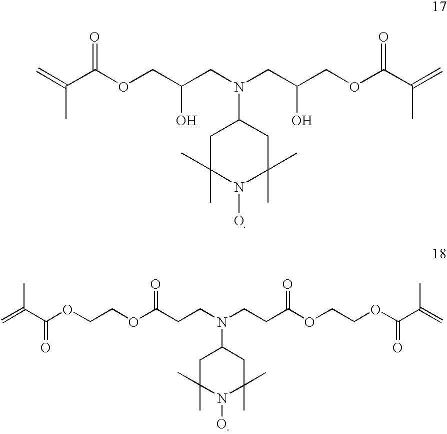

Surprisingly, the addition reaction of diepoxides and the steric hindered 4-amino-2,2,6,6-tetramethylpiperidin (ATMP) leads to linear soluble epoxide-amine addition polymers. The secondary amino groups do not react under the conditions of this polymerization. In the same manner the addition ATMP and Glycidylmethacrylat or Ethylene glycol acrylate methacrylate, respectively results in non-branched macromonomers.

Not less surprisingly it was found that the oxidation of prepolymers, macromonomers and polymers containing ATMP is possible without of a considerable degree of oxidation of hydroxylic moieties or methacrylic groups. The absorptions of hydroxylic groups at 3459/3421 cm⁻¹ and of the double bond at 1637 cm⁻¹ remains unchanged in the IR spectra compared to the non-oxidized molecules. Furthermore, no absorption of a keto group was observed.

The invented dental composition comprises stable radicals of formulas 1 to 5 in a content of 0.001 to 3.0% by weight, preferably in a content of 0.01 to 1.0% by weight and most preferably in a content of 0.1 to 0.5% by weight.

For example a composite containing 2,2-Bis-[p-(2-hydroxy-3-methacryloyloxypropoxy)-phenyl]-propane, Triethyleneglycol dimethacrylate, UDMA, Camphor quinone and N,N-Dimethylaminoethylbenzoic acid ethylester and a Barium-alumo-silicate glass show a light sensitivity of 25 seconds at 10,000 lux. The compressive strength is 343.9±7.3 MPa, the flexural strength (ISO 4049) is 119.2±9.3 MPa and the E-modulus is 7802±293 MPa.

A composite of the same composition that comprises additionally N,N-Bis-(2-hydroxy-3-methacryloyloxypropoxy)-4-amino-2,2,6,6-tetramethylpiperidin-1-oyxl radical of example 1 show a improved light sensitivity of 175 seconds at 10,000 lux.

### EXAMPLE 1

N,N-Bis-(2-hydroxy-3-methacryloyloxypropoxy)4-amino-2,2,6,6-tetramethylpiperidin (GMA-ATMP)

4.998 g (35.17 mmol) Glycidylmethacrylat and 2.754 g (17.59 mmol) 4-amino-2,2,6,6-tetramethylpiperidin were homogeneously mixed and reacted for 48 hours at 80° C. After that time the absorption of epoxide groups at 910 cm⁻¹ is completely missing.

Yield 7.756 g (100% of th.)

C₂₃H₄₀N₂O₆, 440.58 g/mol

IR (cm⁻¹): 3421 (OH), 2975/2935 (CH₂/CH₃), 1726 (CO), 1637 (C═C)

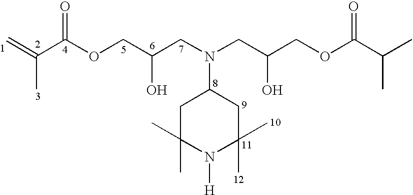

¹³C NMR (ppm): 126.0 (1), 136.0 (2), 18.3 (3), 167.3 (4), 67.7/68.5 (5), 66.7/67.1 (6), 63.1 (7), 54.0/54.2 (8), 51.3/51.8 (9), 41.3 (10), 28.4/28.5 (11), 35.2 (12)

N,N-Bis-(2-hydroxy-3-methacryloyloxypropoxy)-4-amino-2,2,6,6-tetramethylpiperidin-1-oxyl Radical (GMA-ATMPO)

In a three-necked flask equipped with a refluxer, a gas inlet pipe and a stirrer were dissolved 7.19 g (16.32 mmol) GMA-ATMP under stirring and heating to 60° C. Then a stream of nitrogen was passed through this solution for 30 minutes.

In 250 ml Erlenmeyer flask were dissolved under stirring 8.06 g (24.48 mmol) K₃Fe(CN)₆ and 4.95 g (123.65 mmol) NaOH in 180 ml water.

Thereafter the aqueous solution was added to the three-necked flask and stirred intensively for 4 hours at 23° C. The organic phase was separated and washed three times with 80 ml of deionized water and dried over Na₂SO₄. After removing the solvent at 50° C. and an end pressure of 3 mbar the products remains.

In the ESR spectrum a strong signal of nitroxyl radicals was found. Yield 3.95 g (53.3% of th.)

IR (Sub.) cm⁻¹: v(O—H) 3411; vₐₛ(CH₃,CH₂) 2960, 2929; vₛ(CH₃,CH₂) 2850; v(C═O) 1716;v(C═C) 1637; v(C—O) 1173

### EXAMPLE 2

N,N-Bis-(2-hydroxy-3-methacryloyloxypropoxy)4-amino-2,2,6,6-tetramethylpiperidin-1-oxyl Radical (GMA-ATMPO)

1.6600 g (11.68 mmol) Glycidylmethacrylat and 1.0000 g (5.84 mmol) 4-amino-2,2,6,6-tetramethylpiperidin-1oxyl radical were homogeneously mixed and reacted 24 hours at 60° C. and 40 hours at 80° C. After that time the absorption of epoxide groups at 910 cm⁻¹ is completely missing.

In the ESR spectrum a strong signal of nitroxyl radicals was found.

Yield 2.660 g (100% of th.)

C₂₃H₃₉N₂O₇, 455.57 g/mol

IR (cm⁻¹): 3452 (OH), 2975/2935 (CH₂/CH₃), 1728 (CO), 1637 (C═C)

### EXAMPLE 3

Poly-[3,7-dihydroxy-1,9-dioxa-5-aza-(2,2,6,6-tetramethylpiperidine) nonamethylene-1,4-phenylene isopropylidene-1,4-phenylene] (AP-ATMP)

5.0000 g (14.69 mmol) Bis-2,2-[4-(2,3-epoxypropoxy)-phenyl]-propane (DGEBA) and 2.2953 g (14.69 mmol) 4-amino-2,2,6,6-tetramethylpiperidin were slightly heated to 60° C. and mixed homogeneously. Then the mixture was reacted at 60° C. for 24 hours. After that time the absorption of epoxide groups at 915 cm⁻¹ is completely missing.

Yield 7.295 g (100% of th.)

(C₃₁H₄₆N₂O₄,)ₙ, (510.71)ₙ g/mol

¹³C NMR (ppm): 31.0 (1), 41.7 (2), 143.5 (3), 127.7 (4), 113.9 (5), 156.4 (6), 69.9 (7), 68.3/68.7 (8), 54.2/54.4 (9), 50.2 (10), 46.8 (11), 51.0/51.2 (12), 35.1/35.2 (13), 28.4/28.7 (14)

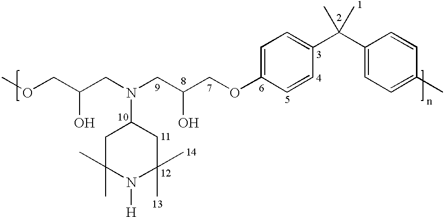

### EXAMPLE 4

In a 250 ml three-necked flask equipped with a refluxer, a gas inlet pipe and a stirrer were dissolved 5.00 g (2.50 mmol) of the steric hindered amine Chimasorb 944 FD (CIBA-Geigy, CAS-Nr. 71878-19-8) in 200 ml Toluene under stirring and heating to 60° C. Then a stream of nitrogen was passed through this solution for 30 minutes.

In 250 ml Erlenmeyer flask were dissolved under stirring 10.70 g (32.50 mmol) K₃Fe(CN)₆ and 6.57 g (164.16 mmol) NaOH in 80 ml water.

Thereafter the aqueous solution was added to the three-necked flask and stirred intensively for 4 hours at 23° C. The organic phase was separated and washed three times with 80 ml of deionized water and dried over Na₂SO₄. After removing the solvent at 50° C. and an end pressure of 3 mbar the products remains.

Yield 4.33 g (86.60% of th.)

In the ESR spectrum a strong signal of nitroxyl radicals was found.

### EXAMPLE 5

N,N-Bis-(3-oxa-4-oxo-6-methacryloyloxyhexyl)-4-amino-2,2,6,6-tetramethylpiperidin (AMA-ATMP)

10.000 g (63.99 mmol) 4-Amino-2,2,6,6-tetramethylpiperidin and 23.57 g (127.98 mmol) Ethylenglycol acrylatmethacrylat were homogeneously mixed and reacted at 23° C. for 14 days. After that time the absorption of acrylate double bond at 1620 cm⁻¹ is completely missing.

Yield 33.57 g (100% of th.)

C₂₃H₄₀N₂O₆, 440.58 g/mol

N,N-Bis-(3-oxa-4-oxo-6-methacryloyloxyhexyl)-4-amino-2,2,6,6-tetramethylpiperidin-1-oxyl radical (AMA-ATMPO)

N,N-Bis-(3-oxa-4-oxo-6-methacryloyloxyhexyl)-4-amino-2,2,6,6-tetramethylpiperidin was oxi-dized according the same procedure as described in example 1.

Yield 5.27 g (97.8% of th.)

In the ESR spectrum a strong signal of nitroxyl radicals was found.

### EXAMPLE 6

N,N-Bis-(3-oxa-4-oxo-6-methacryloyloxyhexyl)-4-amino-2,2,6,6-tetramethylpiperidin-1-oxyl Radical (AMA-ATMPO)

1.075 g (5.84 mmol) Ethylenglycol acrylatmethacrylat and 1.0000 g (5.84 mmol) 4-Amino-2,2,6,6-tetramethylpiperidin-1 oxyl radical were homogeneously mixed and reacted 24 hours at 60° C. and 40 hours at 80° C. After that time the absorption of acrylate double bond at 1620 cm⁻¹ is completely missing.

In the ESR spectrum a strong signal of nitroxyl radicals was found.

Yield 2.075 g (100% of th.)

C₂₇H₄₃N₂O₉, 539.65 g/mol

IR (cm⁻¹): 2960/2845 (CH₂/CH₃), 1720 (CO), 1637 (C═C)

### COMPARATIVE EXAMPLE 1

39.742 g 2,2-Bis-[p-(2-hydroxy-3-methacryloyloxypropoxy)-phenyl]-propane, 24.839 g Triethyleneglycol dimethacrylate, 34.774 g Urethane dimethacrylate, 0.298 g chamfer quinone and 0.348 g Dimethylaminoethyl benzoic acid ethylester were mixed homogeneously. To this resin mixture were added 270.370 g of a barium alumo-silicate glass and mixed homogeneously.

The properties are summarized in Table 1.

### APPLICATION EXAMPLE 1

39.742 g 2,2-Bis-[p-(2-hydroxy-3-methacryloyloxypropoxy)-phenyl]-propane, 24.839 g Triethyleneglycol dimethacrylate, 34.774 g Urethane dimethacrylate, 0.298 g chamfer quinone, 0.348 g Dimethylaminoethyl benzoic acid ethylester and 0.034 g 4-Amino-2,2,6,6-tetramethyl-piperidin-1-oxyl radical (Fluka) were mixed homogeneously. To this resin mixture were added 270.370 g of a barium alumo-silicate glass and mixed homogeneously.

The properties are summarized in Table 1.

### APPLICATION EXAMPLE 2

39.742 g 2,2-Bis-[p-(2-hydroxy-3-methacryloyloxypropoxy)-phenyl]-propane, 24.839 g Triethyleneglycol dimethacrylate, 34.774 g Urethane dimethacrylate, 0.298 g chamfer quinone, 0.348 g Dimethylaminoethyl benzoic acid ethylester and 0.091 g GMA-ATMPO of example 2 were mixed homogeneously. To this resin mixture were added 270.370 g of a barium alumo-silicate glass and mixed homogeneously.

The properties are summarized in Table 1.

### APPLICATION EXAMPLE 3

39.742 g 2,2-Bis-[p-(2-hydroxy-3-methacryloyloxypropoxy)-phenyl]-propane, 24.839 g Triethyleneglycol dimethacrylate, 34.774 g Urethane dimethacrylate, 0.298 g chamfer quinone, 0.348 g Dimethylaminoethyl benzoic acid ethylester and 0.100 g AMA-ATMPO of example 5 were mixed homogeneously. To this resin mixture were added 270.370 g of a barium alumo-silicate glass and mixed homogeneously.

The properties are summarized in Table 1.

### APPLICATION EXAMPLE 4

39.742 g 2,2-Bis-[p-(2-hydroxy-3-methacryloyloxypropoxy)-phenyl]-propane, 24.839 g Triethyleneglycol dimethacrylate, 34.774 g Urethane dimethacrylate, 0.298 g chamfer quinone, 0.348 g Dimethylaminoethyl benzoic acid ethylester and 0.100 g of oxidized amine of example 4 were mixed homogeneously. To this resin mixture were added 270.370 g of a barium alumo-silicate glass and mixed homogeneously.

The properties are summarized in Table 1.

TABLE 1
Properties of dental composites of application examples 1 to 3 and of comparative example 1

| | | Example | | | |
|---|---|---|---|---|---|
| | | Comp. 1 | Appl. 1 | Appl. 2 | Appl. 3 |
| Sensitivity to ambient light, ISO 4049 | sec | 25 | 185 | 180 | 180 |
| (10000 lux) | | | | | |
| Compressive strength | MPa | 343.9 ± 7.3 | 318.6 ± 17.8 | 316.3 ± 11.1 | 338.5 ± 6.6 |
| Flexural strength, ISO 4049 | MPa | 119.2 ± 9.3 | 107.7 ± 10.7 | 108.3 ± 5.0 | 117.9 ± 5.6 |
| E-modulus | MPa | 7802 ± 293 | 7691 ± 343 | 7324 ± 442 | 7698 ± 212 |

We claim:

1. Dental composition having an improved light and thermal stability, comprising a mixture of
(i) at least a polymerizable resin
(ii) at least a polymerizable monomer
(iii) at least a polymerization initiator and/or a sensitizer and stabilizer
(vi) at least an organic and/or inorganic filler and pigments in a content of 0 to 90 percent
(vii) and at least one of the stable radicals of formulas 1 to 5 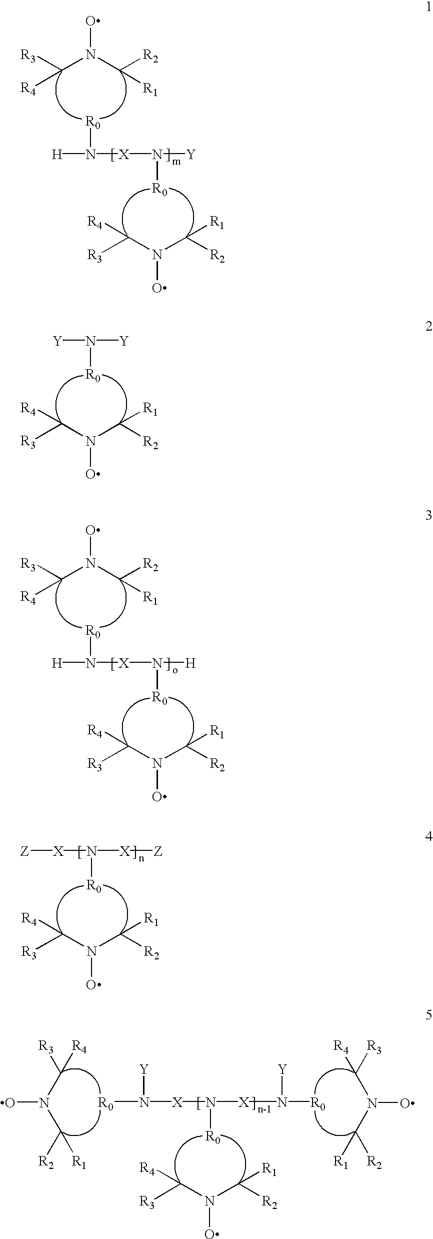
wherein
R₀ denotes a substituted or unsubstituted C₁ to C₁₈ alkylene,
R₁, R₂, R₃ and R₄ denotes a substituted or unsubstituted C₁ to C₁₈ alkylene, preferably a methyl group
X denotes a difunctional substituted or unsubstituted C₂ to C₃₀ alkylene, C₅ to C₃₀ substituted or unsubstituted cycloalkylene, substituted or unsubstituted C₅ to C₃₀ arylene or heteroarylene, preferably the following structures 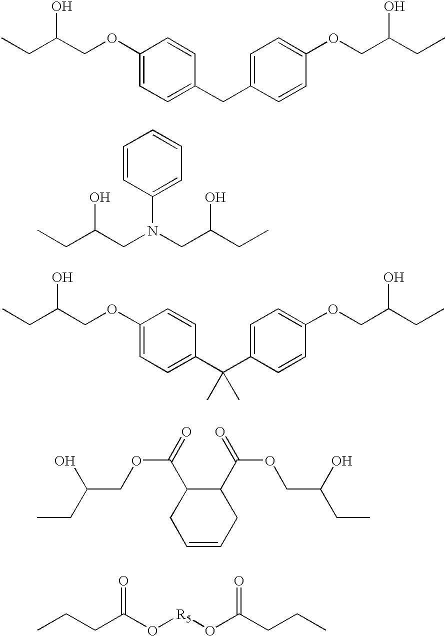
wherein R₅ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
Y denotes H or a monofunctional substituted or unsubstituted C₁ to C₁₈ alkyl, C₅ to C₁₈ substituted or unsubstituted cycloalkyl, substituted or unsubstituted C₅ to C₁₈ aryl or heteroaryl, preferably selected from the group 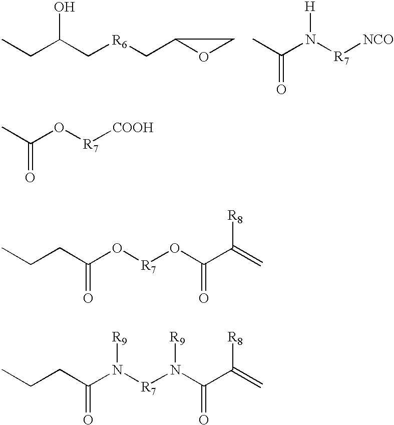
wherein
R₆ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably 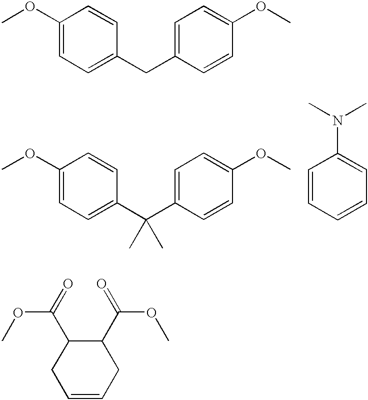
R₇ denotes difunctional substituted or unsubstituted C₁ to C18 alkylene, C₅ to C₁₈ substituted or unsubstituted cycloalkylene, substituted or unsubstituted C₅ to C₁₈ arylene or heteroarylene, preferably selected from the group
R₈ denotes H or a monofunctional substituted or unsubstituted C₁ to C30 alkylene, C₅ to C₃₀ substituted or unsubstituted cycloalkylene, substituted or unsubstituted C₅ to C₃₀ arylene or heteroarylene
R₉ denotes a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
Z denotes hydrogen, or a polymerizable moiety, preferably selected from the group of 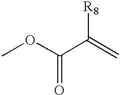
wherein
R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
n, m and o are integers.

2. Dental composition of claim 1, comprising at least one of the compounds 6 to 10 which having at least one piperidinium nitroxyl radical moiety 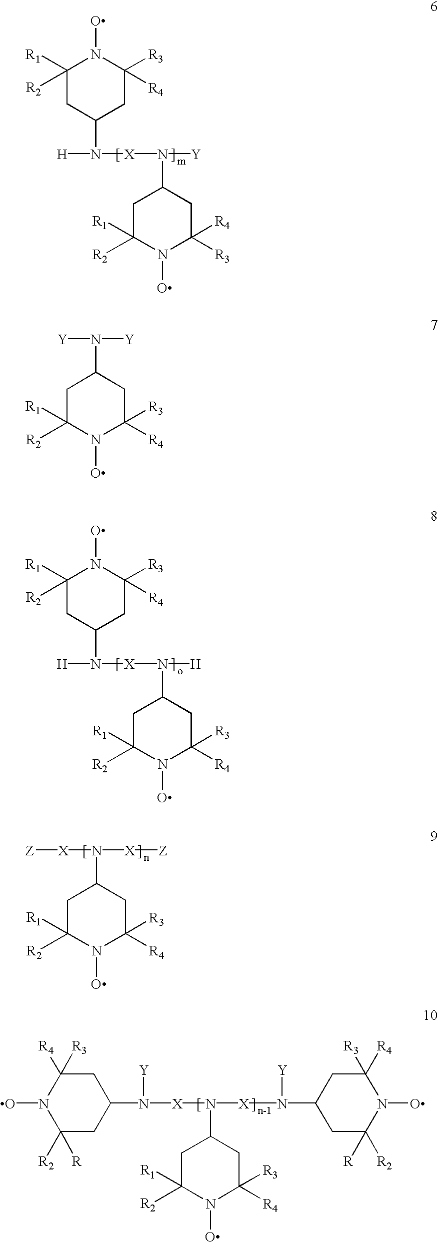
wherein
R₁, R₂, R₃ and R₄ denotes a substituted or unsubstituted C₁ to C₁₈ alkylene, preferably methyl group
X denotes a difunctional substituted or unsubstituted C₂ to C₃₀ alkylene, C₅ to C₃₀ substituted or unsubstituted cycloalkylene, substituted or unsubstituted C₅ to C₃₀ arylene or heteroarylene, preferably the following structures 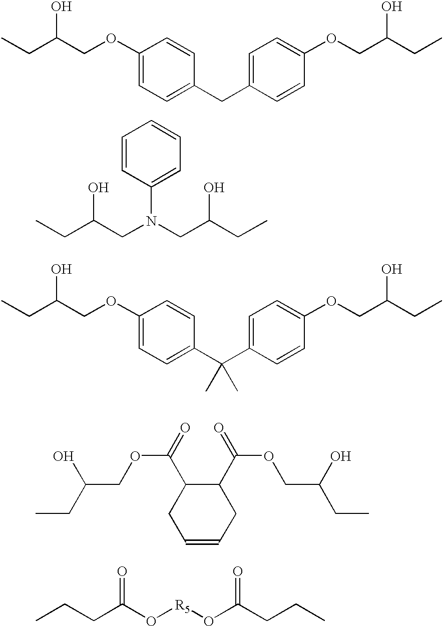
wherein R₅ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
Y denotes H or a monofunctional substituted or unsubstituted C₁ to C₁₈ alkyl, C₅ to C₁₈ substituted or unsubstituted cycloalkyl, substituted or unsubstituted C₅ to C₁₈ aryl or heteroaryl, preferably selected from the group 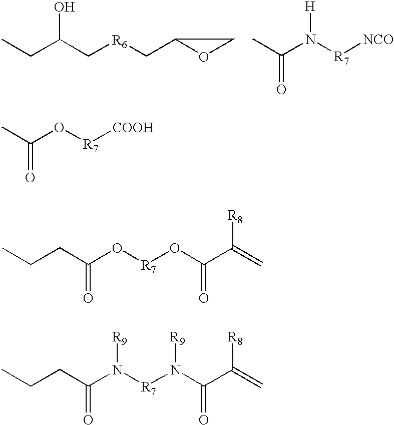
wherein
R₆ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably 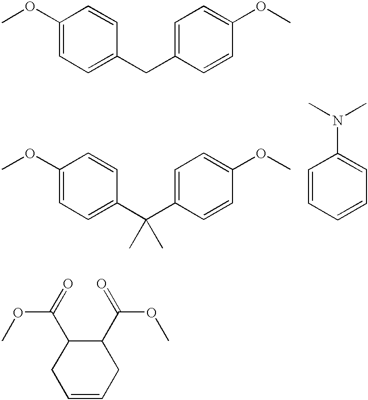
R₇ denotes difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably selected from the group
R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
R₉ denotes a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
Z denotes hydrogen, or a polymerizable moiety, preferably selected from the group of 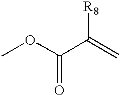
wherein
R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
n, m and o are integers.

3. Dental composition of claims 1 and 2, wherein molecules containing piperidinium nitroxyl radical moieties were obtained by oxidation of the following compounds 11 to 15 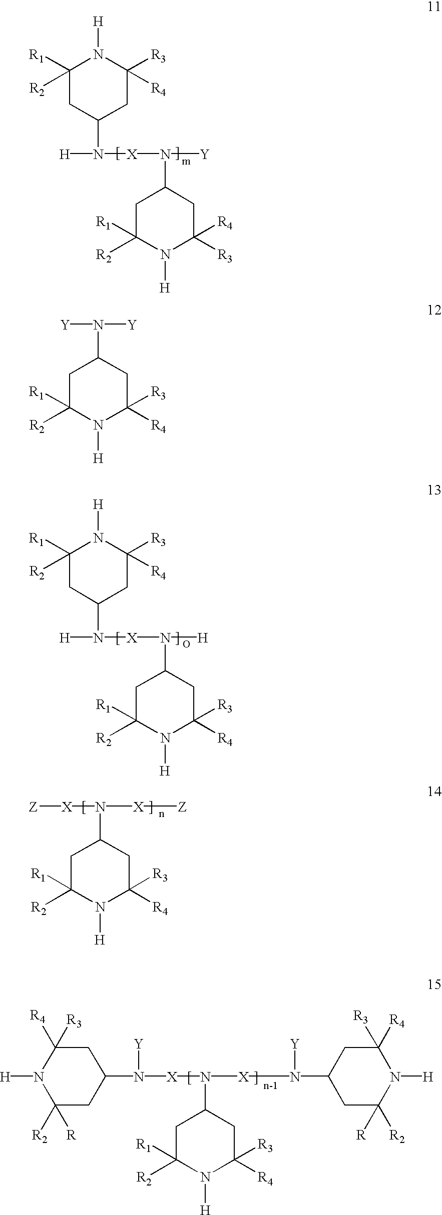
wherein
R₁, R₂, R₃ and R₄ denotes a substituted or unsubstituted C₁ to C₁₈ alkylene, preferably a methyl group
X denotes a difunctional substituted or unsubstituted C₂ to C₃₀ alkylene, C₅ to C₃₀ substituted or unsubstituted cycloalkylene, substituted or unsubstituted C₅ to C₃₀ arylene or heteroarylene, preferably the following structures 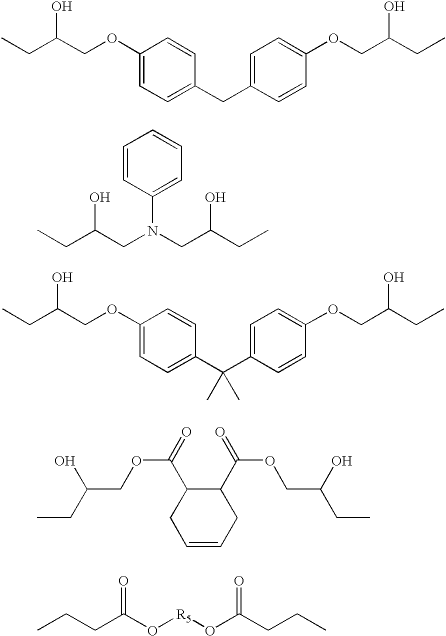
wherein R₅ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene,
Y denotes H or a monofunctional substituted or unsubstituted C₁ to C₁₈ alkyl, C₅ to C₁₈ substituted or unsubstituted cycloalkyl, substituted or unsubstituted C₅ to C₁₈ aryl or heteroaryl, preferably selected from the group 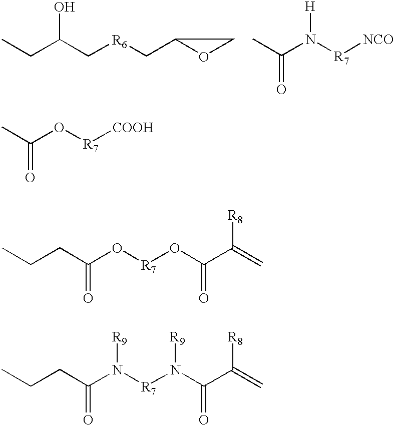
wherein
R₆ denotes a difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably 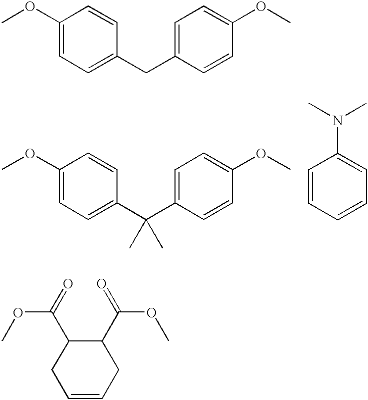
R₇ denotes difunctional substituted or unsubstituted C₁ to C₁₈ alkylene, C₅ to C₁₈ substituted or unsubstituted cycloalkylene, substituted or unsubstituted C₅ to C₁₈ arylene or heteroarylene, preferably selected from the group
R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
R₉ denotes a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
Z denotes hydrogen, or a polymerizable moiety, preferably selected from the group of 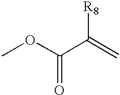
wherein
R₈ denotes H or a monofunctional substituted or unsubstituted C₁ to C₃₀ alkylene, C₅ to C₃₀ substituted or unsubstituted cycloalkylene, substituted or unsubstituted C₅ to C₃₀ arylene or heteroarylene
n, m and o are integers.

4. Dental composition of claim 1, wherein polymers, prepolymers or macromonomers containing nitroxyl radical moieties were obtained by reaction of compound 16 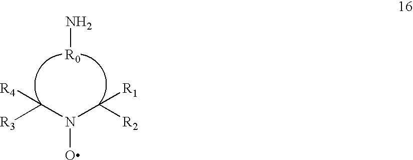
wherein
R₀ denotes a substituted or unsubstituted C₁ to C₁₈ alkylene,
R₁, R₂, R₃ and R₄ denotes a substituted or unsubstituted C₁ to C₁₈ alkylene, preferably methyl group
with a molecule of group A, selected from the group of a diepoxide, a diisocyanate, a dicarboxylic acid or a derivative thereof, a bisacrylamide or a bisacrylate or
with a molecule of group B, selected from the group of molecules that comprise at least an epoxide and a methacrylate group, an epoxide and an isocyanate, a methacrylate and an isocyanate group, an acrylate and a methacrylate group, or
with a mixture of molecules A and B.

5. Dental composition of claim 4, wherein amines containing nitroxyl radical moieties are contained as comonomers in polyamides, polyamidoamines, polyesteramines, polyureas, epoxide-amine addition polymers.

6. Dental composition of claim 4, wherein amines containing nitroxyl radical moieties are contained as comonomers in macromonomers or prepolymers having polyamide, polyamidoamine, polyesteramine, polyurea or epoxide-amine addition polymer structural units.

7. Dental composition of claim 1, wherein molecules containing piperidinium nitroxyl radical preferably are compounds 17 and 18.

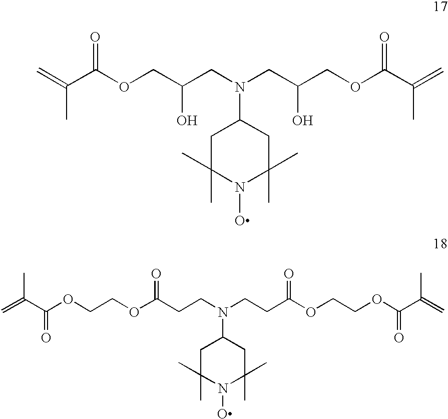

8. Dental composition of claim 1, wherein the dental composition comprises stable radicals of formulas 1 to 5 in a content of 0.001 to 3.0% by weight.

9. Dental composition of claim 1, wherein the dental composition preferably comprises stable radicals of formulas 1 to 5 in a content of 0.01 to 1.0% by weight.

10. Dental composition of claim 1, wherein the dental composition most preferably comprises stable radicals of formulas 1 to 5 in a content of 0.01 to 0.2% by weight.
